(19) United States
(12) Patent Application Publication
(10) Pub. No.: US 2024/0124383 A1
(43) Pub. Date: Apr. 18, 2024

(54) METHODS FOR PRODUCTION OF AN ESTER OF BUTYRIC ACID

(71) Applicant: SUPERBREWED FOOD, INC., New Castle, DE (US)

(72) Inventors: Asher Vitner, Jerusalem (IL); Aharon M. Eyal, Jerusalem (IL); Bryan P. Tracy, Wilmington, DE (US)

(21) Appl. No.: 18/277,092

(22) PCT Filed: Feb. 13, 2022
(86) PCT No.: PCT/IB2022/051264

§ 371 Date: Aug. 14, 2023

Related U.S. Application Data
(60) Provisional application No. 63/150,744, filed on Feb. 18, 2021.

(51) Int. Cl. C07C 67/08; A23K 20/105; A23L 29/00; C07C 67/54

(52) U.S. Cl. CPC C07C 67/08; A23K 20/105; A23L 29/035; C07C 67/54

(57) ABSTRACT

Provided is a method for producing an ester of butyric acid from an acid mixture, comprising contacting an aqueous carboxylic acid mixture comprising butyric acid and at least one additional carboxylic acid selected from the group consisting of formic acid, acetic acid, propionic acid, lactic acid, hydroxybutyric acid and combinations thereof, wherein at least 30% of the total amount of acids in the aqueous carboxylic acid mixture are in free acid form, with a liquid extractant having a Hildebrand solubility parameter of less than 25 MPa^{(1/2)}; separating a resultant butyric acid-comprising extract from a residual aqueous phase; contacting a resultant separated butyric acid-comprising extract with an alkanol in the presence of an acid catalyst, to form a product comprising an ester of butyric acid and alkanol and optionally water; wherein when water is formed, separating water to provide a separated product comprising an ester of butyric acid.

### CROSS-REFERENCE TO RELATED APPLICATION

The present application gains priority from U.S. Provisional Application No. 63/150,744 filed Feb. 18, 2021 which is incorporated by reference as if fully set-forth herein.

### FIELD OF THE INVENTION

The present invention relates to methods of producing esters of butyric acid, and more specifically to methods of producing such esters from an acid mixture by contacting an acid mixture comprising butyric acid and at least one additional carboxylic acid with a liquid extractant having a Hildebrand solubility parameter of less than 25 MPa^{(1/2)}; separating the resultant butyric acid-comprising extract and residual aqueous phase; contacting the separated butyric acid-comprising extract with alkanol in a reactor to form a product comprising an ester of butyric acid and optionally water; and separating any water formed from the product.

### BACKGROUND

Methods for preparation of esters from an acid and an alkanol are known in the art, for example production of glyceride esters of butyric acid from glycerol and synthetic butyric acid. However, production from natural butyric acid presents major difficulties. The natural butyric acid is provided in a relatively dilute fermentation liquor, which contains a salt of the acid, rather than the free acid form. Additionally, the fermentation liquor comprises carboxylic acids, such as acetic and lactic as by-products, which may also react with the alkanol and contaminate the product. Additional contamination may result from fermentation-resulting impurities, such as residual carbohydrates, peptides and colored products of Maillard reactions.

### SUMMARY OF THE INVENTION

According to an aspect of some embodiments of the present invention, there is provided a method for producing an ester of butyric acid from an acid mixture, comprising
(i) providing an aqueous carboxylic acid mixture comprising butyric acid and at least one additional carboxylic acid, wherein said at least one additional carboxylic acid is selected from the group consisting of formic acid, acetic acid, propionic acid, lactic acid, hydroxybutyric acid and combinations thereof, wherein at least 30% wt of a total amount of acids in said aqueous carboxylic acid mixture are in free acid form;
(ii) contacting in an extractor said acid mixture with a liquid extractant having a Hildebrand solubility parameter of less than 25 MPa^{(1/2)}, whereby a butyric acid-comprising extract and a residual aqueous phase are formed;
(iii) separating said extract from said residual aqueous phase, whereby separated butyric acid-comprising extract and separated residual aqueous phase are formed;
(iv) providing an alkanol;
(v) contacting in a reactor said separated butyric acid-comprising extract with said alkanol in the presence of an acid catalyst, whereby a product comprising an ester of butyric acid with said alkanol and optionally water are formed in said reactor; and
(vi) wherein when water is formed, separating water from said product, whereby a separated product comprising an ester of butyric acid is formed.

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a schematic representation of an embodiment of a method in accordance with the principles of the present invention; and

FIG. 2 is a schematic representation of a further embodiment of the method of the present invention.

### DETAILED DESCRIPTION OF THE INVENTION

The present invention relates to methods of producing esters of butyric acid, and more specifically to methods of producing such esters from an acid mixture by contacting an acid mixture comprising butyric acid and at least one additional carboxylic acid with a liquid extractant having a Hildebrand solubility parameter of less than 25 MPa^{(1/2)}; separating the resultant butyric acid-comprising extract and residual aqueous phase; contacting the separated butyric acid-comprising extract with alkanol in a reactor to form a product comprising an ester of butyric acid and optionally water; and separating any water formed from the product.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “aqueous carboxylic acid mixture” refers to an aqueous solution comprising butyric acid and at least one additional acid.

As used herein, the term “refined” with respect to a butyric acid-containing extract means an extract having a ratio of butyric acid to another carboxylic acid which is at least 10% greater than that ratio in the extract prior to refining.

As used herein, the term “refining” refers to one or more of distilling (i.e. removing volatile impurities, including residual extractant); contacting with an adsorbent and/or ion-exchanger; treatment with active carbon (for removal of colour); and combinations thereof.

As used herein, the term “enriched” with respect to a component of a first or second phase of a reactor means having a proportion of the specified component that is at least 10% greater than that present in the overall content of the reactor.

As used herein, the term “continuous mode” with regard to a process as disclosed herein means that steps (ii) to (v) of the process are conducted continuously, such that an acid mixture is continuously provided to the extractor and said product comprising an ester is continuously removed from the reactor.

As used herein, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed. As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

According to an aspect of some embodiments of the present invention, there is provided a method for producing an ester of butyric acid from an acid mixture, comprising
(i) providing an aqueous carboxylic acid mixture comprising butyric acid and at least one additional carboxylic acid, wherein said at least one additional carboxylic acid is selected from the group consisting of formic acid, acetic acid, propionic acid, lactic acid, hydroxybutyric acid and combinations thereof, wherein at least 30% wt of a total amount of acids in said aqueous carboxylic acid mixture are in free acid form;
(ii) contacting in an extractor said acid mixture with a liquid extractant having a Hildebrand solubility parameter of less than 25 MPa^{(1/2)}, whereby a butyric acid-comprising extract and a residual aqueous phase are formed;
(iii) separating said extract from said residual aqueous phase, whereby separated butyric acid-comprising extract and separated residual aqueous phase are formed;
(iv) providing an alkanol;
(v) contacting in a reactor said separated butyric acid-comprising extract with said alkanol in the presence of an acid catalyst, whereby a product comprising an ester of butyric acid with said alkanol and optionally water are formed in said reactor; and wherein when water is formed, separating water from said product, whereby a separated product comprising an ester of butyric acid is formed

According to an embodiment, at least 90% of said extract, such as 90%, 91%, 92%, 93%, 94%, 95%, 96%, 97%, 98%, 99% or even 100% is separated from said residual aqueous phase.

According to an embodiment, the total amount of acids of the aqueous carboxylic acid mixture may be present at any concentration. According to an embodiment, the combined concentration of butyric acid and the at least one additional acid is at least 5% wt, 10% wt, 15% wt, 20% wt, 25% wt, 30% wt, 35% wt, 40% wt, 45% wt or even at least 50% wt of the total aqueous carboxylic acid mixture.

According to an embodiment, the butyricacid and the at least one additional acid are present in the mixture at any weight/weight ratio. According to an embodiment, the weight/weight ratio between the butyric acid and the at least one additional acid in the mixture is at least 1.0, 1.5, 2.0, 2.5 or 3.0.

According to an embodiment, any carboxylic acid may be used as the additional carboxylic acid. According to an embodiment, the additional carboxylic acid comprises at least 60%, at least 70%, at least 80%, at least 90%, or even at least 95% acetic acid. According to an embodiment, the additional carboxylic acid comprises acetic acid and lactic acid.

According to an embodiment, the method comprises refining said separated extract, wherein a refined extract is formed.

According to an embodiment, the method comprises contacting in said reactor said separated extract or said refined extract with said alkanol in the presence of an acid catalyst, wherein a product comprising an ester of butyric acid with said alkanol is formed in said reactor.

According to an embodiment, said acid mixture comprises at least one non-acid impurity. According to such an embodiment, a ratio of said at least one non-acid impurity to said ester in said product comprising an ester is less than a ratio of said impurity to said acid in said acid mixture. According to an embodiment, said at least one non-acid impurity is selected from the group consisting of carbohydrates, amino acids, mineral salts and combinations thereof.

According to an embodiment, said providing an aqueous acid mixture comprises providing an aqueous solution having a pH in the range of from about 5 to about 8 comprising a salt of said butyric acid and a salt of said at least one additional carboxylic acid with at least one counter ion, reacting said aqueous solution with a mineral acid, whereby a salt of said mineral acid and said at least counter ion is formed and separating said salt. According to such an embodiment, said mineral acid comprises sulfuric acid, said counter ion comprises ammonium, said formed salt comprises (NH₄)2SO₄ and wherein said separating the salt comprises crystallizing it to form (NH₄)2SO₄ crystals and separating said (NH₄)2SO₄ crystals. According to one such embodiment, said aqueous acid mixture is saturated with (NH₄)2SO₄.

According to an embodiment, the amount of mineral acid reacted with said aqueous solution is equivalent to the total amount of carboxylic acids in the aqueous acid mixture (i.e. one equivalent of the mineral acid per mole of the total amount of carboxylic acids).

According to an embodiment, addition of the mineral acid lowers the pH of the reaction solution to a value less than that of the pKa value of the carboxylic acids (such as pH less than 4.0, 3.5, 3.0 or 2.5) and at least partially converts the salt form of the carboxylic acid (which is present in the solution at pH greater than that of the pKa value of the carboxylic acids) to the free carboxylic acid form.

According to an embodiment, at least 30%, 35%, 40%, 45%, 50%, 55%, 60%, 65%, 70%, 75%, 80%, 85%, 90%, 95% or even 100% of a total amount of carboxylic acids in said aqueous carboxylic acid mixture are in free acid form.

According to an embodiment, said counter ion is selected from the group consisting of sodium, ammonium, potassium, calcium, magnesium and combinations thereof.

According to an embodiment, said mineral acid is selected from the group consisting of sulfuric acid, hydrochloric acid, phosphoric acid, nitric acid and combinations thereof.

In embodiments wherein said mineral acid comprises sulfuric acid, the sulfuric acid is added to the neutral aqueous solution in an amount sufficient to reduce the pH as disclosed above, but preferably not lower than 2.5, such that the sulfate mostly stays in the form of SO₄, rather than HSO₄ and the formed salt is (NH₄)2SO₄ rather than NH₄HSO₄.

According to an embodiment, the concentration of the formed salt in the neutral aqueous solution is sufficiently high for the salt to reach saturation level, such that crystals of the salt are formed. According to an embodiment, the aqueous solution is concentrated prior to, simultaneously with or after the addition of the mineral acid, such that at least 50% wt, 60% wt, 70% wt, 80% wt or at least 90% wt of the salt crystallizes out. The final concentration of the solution required in order to obtain crystallization depends on the nature and solubility of the formed salt. According to an embodiment, the concentration of the butyrate salt in said provided aqueous solution having about neutral pH is between about 5% wt and 20% wt and said mineral acid is added without concentrating prior to or simultaneously with the addition of the mineral acid. According to an embodiment, concentrating is conducted on said separated residual aqueous phase after separating from said extract.

According to an embodiment, the crystallized salt is separated from the aqueous solution by filtration and/or centrifugation.

According to an embodiment, said providing said aqueous solution comprises fermenting a carbohydrate with a butyric acid forming microorganism.

According to an embodiment, any butyric acid-forming microorganism is suitable.

According to an embodiment, said microorganism comprises a microorganism of the genus Clostridium. According to one such embodiment, the microorganism comprises Clostridium tyrobutyricum.

According to an embodiment, said neutral aqueous solution is formed upon culturing the microorganism in an aqueous solution comprising said carbohydrate, followed by filtering out the formed biomass. According to one such embodiment, the filtered out biomass is used as a single cell protein.

According to an embodiment, said non-acid impurities in said acid mixture comprise impurities resulting from fermentation, e.g. residual carbohydrates and/or residual fermentation micro-nutrients, such as amino acids. According to an embodiment, said non-acid impurities are mostly hydrophilic compounds.

According to an embodiment, said butyric acid-containing extract further comprises at least one of said additional carboxylic acids, and said refining comprises contacting said butyric acid-containing extract with water, whereby at least a fraction, such as at least 50%, at least 60%, at least 70%, at least 80%, at least 90% or even 100% of said additional carboxylic acid is washed out to form said refined extract.

According to an embodiment, said contacting of said provided acid mixture with said liquid extractant is carried out in an industrial extractor, e.g. in an extraction column or an extraction mixer-settler. According to an embodiment, said contacting is carried out in a counter-current mode and comprises 1-10 theoretical stages.

According to an embodiment, the extractant has a Hildebrand solubility parameter of less than 25, 24, 23, 22, 21, 20 or even less than 19 MPa^{(1/2)}. According to an embodiment, the extractant has a Hildebrand solubility parameter of more than 16, 17 or even more than 18 MPa^{(1/2)}.

According to an embodiment, the butyric acid-containing extract comprises at least 85% wt, 90%, 95%, 98% or even at least 99% wt of the butyric acid in said aqueous mixture.

According to an embodiment, in addition to formation of said butyric acid-containing extract, said contacting said aqueous acid mixture with said liquid extractant forms a residual aqueous phase. According to one such embodiment, said residual aqueous phase comprises at least a fraction (at least 50% wt, 60%, 70%, 80%, 90% or 95%) of said additional carboxylic acid in said aqueous mixture.

According to an embodiment wherein said provided acid mixture is a product of reacting a neutral aqueous solution with a mineral acid, said residual aqueous phase comprises said formed salt of said mineral acid and said counter ion.

According to an embodiment, the extractant comprises, in addition to butyric acid, at least one additional carboxylic acid. According to an embodiment, a weight/weight ratio of said butyric acid to said additional carboxylic acid in the extract is greater than 3, 5, 7, 9, 11, 13 or 15. According to an embodiment, a weight/weight ratio of said butyric acid to said additional carboxylic acid in the extract is greater than a weight/weight ratio of said butyric acid to said additional carboxylic acid in the aqueous acid mixture by a factor of 2, 3, 4, 5, 6, 7, 8, 9, or 10.

According to an embodiment, said refining comprises contacting said extract with water, whereby at least a fraction of said additional carboxylic acid is washed out to form said refined extract. According to an embodiment, a weight/weight ratio of said butyric acid to said additional carboxylic acid in said refined extract is greater than a weight/weight ratio of said butyric acid to said additional carboxylic acid in the extract by a factor of 2, 3, 4, 5, 6, 7, 8, 9, or 10.

According to an embodiment, said extractant comprises toluene.

According to an embodiment, said method comprises contacting in a reactor said separated butyric acid-comprising extract with said alkanol in the presence of an acid catalyst, whereby a product comprising an ester of butyric acid with said alkanol and optionally water are formed in said reactor. According to an embodiment, the amount of alkanol is about one mole of alkanol per mole of butyric acid in said extract. According to an embodiment, said alkanol is selected from the group consisting of C1-C4 mono- to tri-ols and esters thereof.

According to an embodiment, said contacting said butyric acid-comprising extract or refined extract with said alkanol is conducted in a reactor equipped with stirring and heating devices and optionally operating at sub-atmospheric pressure. According to an embodiment, said reactor is contacted with a distillation column.

According to an embodiment, said contacting said butyric acid-comprising extract or refined extract with said alkanol is conducted in the presence of an acid catalyst, e.g. a mineral acid added to the reactor or a resin carrying sulfate moieties, such as in strong acid cation exchangers.

According to an embodiment, said contacting said butyric acid-comprising extract or refined extract with said alkanol produces said product comprising an ester of butyric acid with said alkanol and water and said reactor is equipped with means for separating water from said product.

According to an embodiment, said alkanol comprises glycerol and said product comprising an ester comprises at least one selected from the group consisting of glycerol mono-butyrate, glycerol di-butyrate and glycerol tri-butyrate and combinations thereof. According to an embodiment, about one third of a mole of glycerol is used per mole of butyric acid in said extract.

According to an embodiment, said product comprising an ester comprises at least two selected from the group consisting of glycerol mono-butyrate, glycerol di-butyrate and glycerol tri-butyrate. According to an embodiment, said at least two comprise glycerol tri-butyrate, such as glycerol tri-butyrate and glycerol mono-butyrate or glycerol tri-butyrate and glycerol di-butyrate. According to an embodiment, said product comprises all three of glycerol mono-butyrate, glycerol di-butyrate and glycerol tri-butyrate.

According to an embodiment, said product comprising an ester comprises at least 50% 60%, 70%, 80%, 90% or even at least 95% glycerol tri-butyrate.

According to an embodiment, said alkanol is an ester of glycerol with a fatty acid and said product comprising an ester comprises said fatty acid.

According to an embodiment, said alkanol is a medium chain triglycerides carrying three C₈-C₁₀ fatty acids and said product comprising an ester carries 1-2 butyric acids and 1-2 fatty acids.

According to an embodiment, said alkanol is selected from the group consisting of methanol, ethanol, propanol, butanol, glycerol and combinations thereof. According to an embodiment, said alkanol is glycerol.

According to an embodiment, said alkanol is provided in free form, e.g. ethanol or glycerol. In such embodiments, the reaction between butyric and the alkanol is an esterification reaction.

According to an alternative embodiment, said alkanol is provided in an ester form, e.g. ethyl acetate or ono-, di- or tri-glyceride. In such embodiments, the reaction between butyric and the alkanol is a trans-esterification reaction, wherein butyric acid replaces the additional acid on the alkanol.

According to an embodiment, said separating water comprises water distillation.

According to an embodiment, said extractant is capable of forming an azeotrope with water and said separating water comprises water-extractant azeotropic distillation.

According to an embodiment, the temperature in said reactor is about the temperature of boiling of said azeotrope at the operating pressure of the reactor.

According to an embodiment, said azeotrope distillation forms vapors comprising water and said extractant. According to an embodiment, said vapors are cooled, whereby two phases are formed, a water phase and an extractant phase, as extractants having the selected Hildebrand solubility parameter have low solubility in water (less than 1%, 0.8%, 0.6%, 0.4% or 0.2%). According to an embodiment, said water phase is separated from said extractant phase and the latter is recycled, at least partially, to said contacting with said acid mixture.

According to an embodiment, on said contacting in a reactor and said forming a product comprising an ester, two phases are formed in the reactor, wherein a first of said phases is more hydrophobic than a second of said phases.

According to an embodiment, the reactor contains hydrophilic compounds, such as the alkanol and any water formed in the reaction, if provided in alkanol form. It may also contain additional hydrophobic compounds, e.g. the extractant, product comprising an ester and the alkanol, if provided in an ester form. According to an embodiment, the relative proportion and the relative solubility of the components in the reactor is such that it provides mutual solubility and a single phase is formed. According to an alternative embodiment, two phases are formed. According to an embodiment, first two phases are formed and then, as more of the alkanol is converted to the product comprising an ester, only one phase is present in the reactor.

According to an embodiment, said first phase is enriched with said product comprising an ester.

According to an embodiment, said second phase is enriched with said alkanol and optionally also enriched with water.

According to an embodiment, said separating water from said ester comprises separating said first phase from said second phase.

According to an embodiment, said contacting in a reactor and said separating water from said ester are conducted in a continuous mode.

According to an embodiment, the method further comprises refining said separated product comprising an ester of butyric acid, whereby a refined product comprising an ester of butyric acid is formed.

According to an embodiment, said separated product comprising an ester of butyric acid comprises residual unreacted alkanol and said refining comprises wash with water, whereby said refined products ester and washed alkanol are formed. According to an embodiment, said washed alkanol is recycled back to the reaction and further contacted with said separated butyric acid-comprising extract.

According to an embodiment, said separated product comprises impurities and said refined product comprises less impurities, such as 10% wt less, 20%, 30%, 40%, 50%, 60%, 70%, 80%, 90% or even 100 wt % less.

According to an aspect of some embodiments of the present invention, there is provided a formulation comprising a separated product comprising an ester of butyric acid as disclosed herein. According to an embodiment, the formulation comprises an encapsulated product of an ester of butyric acid, such as a microcapsule.

According to an aspect of some embodiments of the present invention, there is provided a formulation comprising a refined product comprising an ester of butyric acid as disclosed herein. According to an embodiment, the formulation comprises an encapsulated refined product of an ester of butyric acid, such as a microcapsule.

According to an embodiment, any one of the formulations disclosed herein optionally further comprises at least one carrier and/or excipient, such as emulsifiers, poly-unsaturated fatty acids (such as omega-3, lipophilic vitamins, carotenoids and the like).

According to an embodiment, the formulation is provided in the form of tablets or capsules.

According to an embodiment, the formulation is for administration to an animal in the form of a food (such as a food intended for human consumption or an animal feed) or a vitamin-containing formulation.

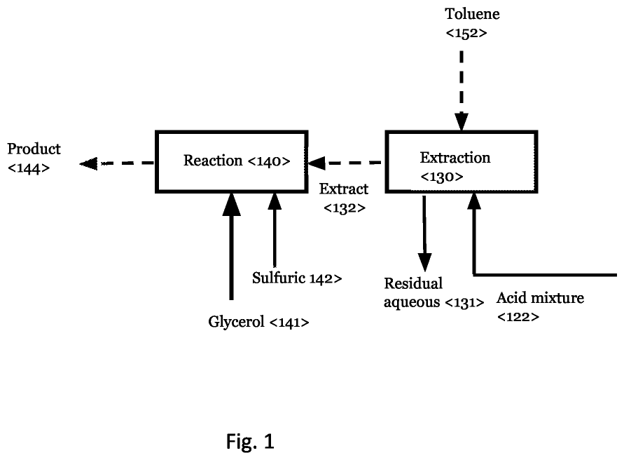

Referring now to FIG. 1, there is schematically presented an embodiment of a method in accordance with the principles of the present invention. The method comprises providing an aqueous carboxylic acid mixture <122> comprising butyric acid and at least one additional carboxylic acid comprising acetic acid, wherein at least 30% wt of a total amount of acids in said aqueous carboxylic acid mixture are in free acid form; contacting acid mixture <122> in an extractor <130> with a liquid extractant comprising toluene <152>, whereby a butyric acid-comprising extract <132> and a residual aqueous phase <131> are formed.

Extract <132> is separated from residual aqueous phase <131>, whereby separated butyric acid-comprising extract and separated residual aqueous phase are formed. An alkanol comprising glycerol <141> is provided and contacted in a reactor <140> with separated butyric acid-comprising extract in the presence of sulfuric acid as an acid catalyst <142>, whereby a product <144> comprising tributyrin glycerol (an ester of butyric acid with alkanol) is formed.

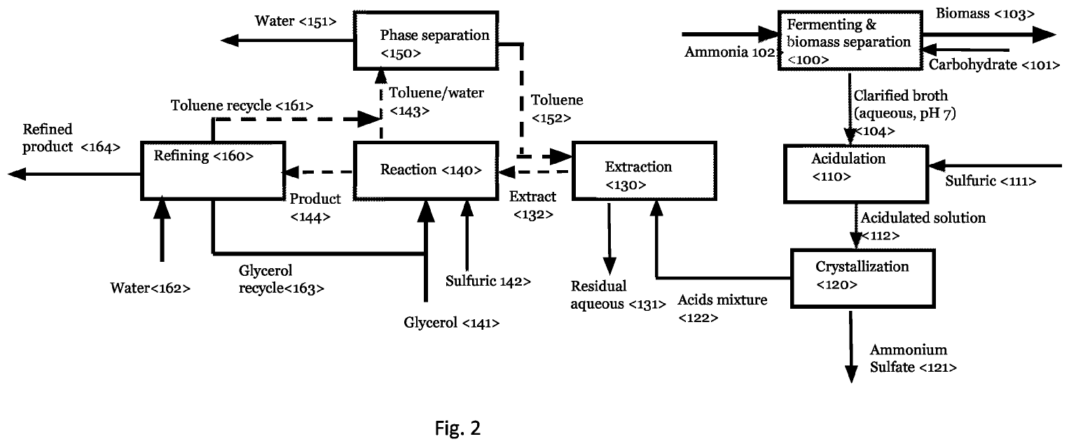

Referring now to FIG. 2, there is shown an embodiment of the method of the present invention wherein aqueous carboxylic acid mixture <122> is produced by fermentation <100> of a carbohydrate <101> with a butyric acid-forming microorganism, wherein a biomass is formed. During fermentation, the pH of the fermentation liquor is maintained at a pH of about 7 using ammonia <102>. Once fermentation is complete, the biomass is separated from the fermentation liquor by microfiltration, forming separated biomass <103> and clarified fermentation liquor <104>, which is a neutral aqueous solution containing ammonium butyrate and ammonium acetate. Sulfuric acid <111> is added to the neutral aqueous solution in an acidulation vessel <110> to reach pH of 2.5. The acidulated solution <112> is treated in a crystallizer <120> for crystallization and separation of ammonium sulfate crystals <121> and for the formation of carboxylic acid mixture <122>.

Carboxylic acid mixture <122>, is contacted in extractor <130> with toluene <152> recycled from a later step (all toluene-containing streams are shown in broken lines) to form extract <132> and residual aqueous solution <131> which are then separated.

Extract <132> contains more than 90% of the butyric acid in the carboxylic acid mixture <122>, while residual aqueous solution <131> contains more than 90% of the acetic acid in said mixture.

Butyric acid-comprising extract <132> is contacted in a reactor <140> at about 90° C. with glycerol <141> in the presence of sulfuric acid <142>, whereby tributyrin glycerol and water are formed. At the temperature of the reaction, vapors containing water and toluene <143> are formed. Those vapors are cooled to provide phase separation <150>, forming two separate phases, water <151> and toluene <152>. The latter is recycled to the extractor <130>. A product comprising tributyrin glycerol <144> is separated from the reactor and refined <160>. Refining comprises distilling off toluene <161>, which is recycled to phase separation and to extraction. Refining further comprises washing out unreacted glycerol with water <162>, whereby an glycerol aqueous solution <163> is formed and recycled to the reaction. A refined product comprising tributyrin glycerol <164> is formed.

### EXAMPLES

### Example 1

A dextrose solution was fermented with a Clostridium strain at a temperature of 30° C. for 20 hours to produce a biomass. Ammonia was used for pH control. At the end of the fermentation period, the biomass was filtered out to form a filtrate comprising ammonium butyrate. The filtrate was treated for water removal and the resultant concentrated filtrate was analyzed. The concentrated filtrate was found to contain 360 g/Kg ammonium butyrate, 140 gr/Kg ammonium acetate, 4 gr/Kg ammonium hydroxybutyrate, 20 gr/Kg crude protein, 7 gr/Kg of amino acids and 19 gr/Kg ash, including 1.9 gr/Kg phosphate. The concentrated filtrate had a deep brown color.

### Example 2

Concentrated sulfuric acid was slowly added to 650 gr of the concentrated filtrate obtained in Example 1, having a neutral pH, with mixing and pH control. Addition of the acid was stopped when a pH of the concentrated filtrate solution reached 2.5. A total of about 140 gr concentrated sulfuric acid was added, which also resulted in warming up of the solution. Formation of ammonium sulfate crystals was observed. The solution was cooled down to ambient temperature and the crystals were filtered off to provide an acid mixture comprising a mixture of about 30% wt butyric acid, 11% wt acetic acid and 0.3% wt hydroxybutyric acid, all three being fully converted to the free acid form. The acid mixture was saturated with ammonium sulfate and further contained crude protein, amino acids and phosphate.

### Example 3

520 gr of toluene was added to 600 gr of the saturated acid mixture from Example 2 at ambient temperature. Mixing was applied for 10 minutes. Then the mixture was allowed to separate into a light lipophilic phases (extract) and a heavy hydrophilic phase (aqueous residue). The two phases were analyzed for their butyric acid and acetic acid content. The results, the calculated distribution coefficients and the calculated selectivity are summarized in Table 1 below.

TABLE 1

| | Butyric | Acetic | Butyric/acetic |
|---|---|---|---|
| | acid (% wt) | acid (% wt) | weight/weight ratio |
| Extract | 28.7 | 2.2% | 13.1 |
| Aqueous residue | 4.2 | 8.9 | 0.47 |
| Distribution | 6.8 | 0.25 | |
| coefficients | | | |
| Butyric/acetic | | | 27 |
| selectivity | | | |

### Example 4

The extract from Example 3 was washed by mixing with 500 gr water for 10 minutes to form a washed extract. The washed extract was allowed to separate into two phases, forming a wash solution and refined extract. The refined extract was analyzed and was found to contain 25% wt butyric acid and 0.3% acetic acid. Butyric to acetic weight/weight ratio in the refined extract was 30 times greater than that in the acid mixture from Example 2. washing.

### Example 5

432.5 gr of the refined extract of Example 4 and 45 gr glycerol were introduced into a reaction bulb of a Dean Stark apparatus, forming two phasese. Mixing and heating were applied. The temperature reached about 97° C. Formed vapors were cooled and condensed into two phases, a light phase (essentially toluene), and a heavy phase (mainly water). The water was removed, while the toluene was recycled to the reaction bulb. Heating was continued for 7 hours.

The formed liquid was then removed from the reaction bulb and was allowed to cool to ambient temperature. The cooled liquid was then refined by washing with 50 gr water in order to remove residual glycerol, providing a glycerol-depleted liquid. The glycerol-depleted liquid was further refined by treating with 50 gr of granular active carbon followed by toluene distillation.

The refined product, which had a yellow color, was analyzed on an HPLC and found to contain 146 gr glycerol esters. Overall reaction yield calculated for butyric acid was about 85%.

1-25. (canceled)

26. A method for producing a separated product comprising an ester of butyric acid from an acid mixture, comprising
(i) providing an aqueous carboxylic acid mixture comprising butyric acid and at least one additional carboxylic acid, wherein said at least one additional carboxylic acid is selected from the group consisting of formic acid, acetic acid, propionic acid, lactic acid, hydroxybutyric acid and combinations thereof, wherein at least 30% wt of a total amount of acids in said aqueous carboxylic acid mixture are in free acid form;
(ii) contacting in an extractor said acid mixture with a liquid extractant having a Hildebrand solubility parameter of less than 25 MPa^{(1/2)}, whereby a butyric acid-comprising extract and a residual aqueous phase are formed;
(iii) separating said extract from said residual aqueous phase, whereby separated butyric acid-comprising extract and separated residual aqueous phase are formed;
(iv) providing an alkanol;
(v) contacting in a reactor said separated butyric acid-comprising extract with said alkanol in the presence of an acid catalyst, whereby a product comprising an ester of butyric acid with said alkanol and optionally water are formed in said reactor; and
(vi) wherein when water is formed, separating water from said product comprising an ester, whereby the separated product comprising an ester of butyric acid is formed.

27. The method of claim 26, comprising refining said separated extract, wherein a refined extract is formed and optionally contacting in said reactor said refined extract with said alkanol in the presence of an acid catalyst, wherein a product comprising an ester of butyric acid with said alkanol is formed in said reactor.

28. The method of claim 26, wherein said acid mixture comprises at least one non-acid impurity, optionally wherein a ratio of said at least one non-acid impurity to said ester in said product comprising an ester is less than a ratio of said impurity to said acid in said acid mixture.

29. The method of claim 26, wherein said providing an aqueous acid mixture comprises providing an aqueous solution having a pH in the range of from about 5 to about 8 comprising a salt of said butyric acid and a salt of said at least one additional carboxylic acid with at least one counter ion, reacting said aqueous solution with a mineral acid, whereby a salt of said mineral acid and said at least one counter ion is formed and separating said salt, optionally wherein said providing said aqueous solution comprises fermenting a carbohydrate with a butyric acid forming microorganism.

30. The method of claim 29, wherein said mineral acid comprises sulfuric acid, said counter ion comprises ammonium, said formed salt comprises (NH4)2SO4 and wherein said separating the salt comprises crystallizing it to form (NH4)2SO4 crystals and separating said (NH4)2SO4 crystals.

31. The method of claim 26, wherein said butyric acid-containing extract further comprises at least one of said additional carboxylic acids, wherein said refining comprises contacting said butyric acid-containing extract with water, whereby at least a fraction of said additional carboxylic acid is washed out to form said refined extract.

32. The method of claim 26, wherein said extractant comprises toluene.

33. The method of claim 26, wherein said alkanol is selected from the group consisting of C1-C4 mono- to tri-ols and esters thereof.

34. The method of claim 26, wherein said alkanol comprises glycerol and wherein said product comprising an ester comprises at least one selected from the group consisting of glycerol mono-butyrate, glycerol di-butyrate and glycerol tri-butyrate and combinations thereof.

35. The method of claim 26, wherein said separating water comprises water distillation.

36. The method of claim 26, wherein said extractant is capable of forming an azeotrope with water and said separating water comprises water-extractant azeotropic distillation.

37. The method of claim 26, wherein on said contacting in a reactor and said forming a product comprising an ester, two phases are formed in the reactor, wherein a first of said phases is more hydrophobic than a second of said phases.

38. The method of claim 37, wherein said first phase is enriched with said product comprising an ester.

39. The method of claim 37, wherein said second phase is enriched with said alkanol and optionally also enriched with water.

40. The method of claim 37, wherein said separating water from said ester comprises separating said first phase from said second phase.

41. The method of claim 26, wherein said contacting in a reactor and said separating water from said ester are conducted in a continuous mode.

42. The method of claim 26, further comprising refining said separated product comprising an ester of butyric acid, whereby a refined product comprising an ester of butyric acid is formed.

43. The method of claim 42, wherein said separated product comprising an ester of butyric acid comprises residual unreacted alkanol and wherein said refining comprises wash with water, whereby said refined products ester and washed alkanol are formed.

44. A formulation comprising a separated product comprising an ester of butyric acid produced according to the method of claim 26, optionally wherein said separated product is an encapsulated product.

45. A formulation comprising a refined product comprising an ester of butyric acid produced according to the method of claim 42, optionally wherein said refined product is an encapsulated product.
